(19) United States
(12) Patent Application Publication
(10) Pub. No.: US 2016/0220836 A1
(43) Pub. Date: Aug. 4, 2016

(54) DEVICE AND METHOD OF PHASE-LOCKING BRAIN STIMULATION TO ELECTROENCEPHALOGRAPHIC RHYTHMS

(71) Applicant: Board of Trustees of the University of Arkansas, Little Rock, AR (US)

(72) Inventor: Nathan Allen Parks, Springdale, AR (US)

(21) Appl. No.: 14/988,363

(22) Filed: Jan. 5, 2016

Related U.S. Application Data
(60) Provisional application No. 62/109,982, filed on Jan. 30, 2015.

(51) Int. Cl. A61N 2/00; A61B 5/04; A61N 2/02; A61B 5/0476; A61N 1/20; A61N 1/36

(52) U.S. Cl. CPC A61N 2/006; A61N 1/20; A61N 1/36025; A61N 2/02; A61B 5/0476; A61B 5/04012

(57) ABSTRACT

The device and method for phase-locking brain stimulation to electroencephalographic rhythms improves the accuracy, specificity, and effectiveness of non-invasive brain stimulation devices by timing pulses of brain stimulation to occur in synchrony with naturally occurring brain rhythms measured at the scalp of a patient in order to treat an assortment of neurological and psychiatric conditions. The device and method provided herein improve non-invasive brain stimulation techniques by time-locking the onset of brain stimulation to the phase of naturally-occurring rhythmic oscillations of brain activity that can be recorded with electroencephalography (EEG). The device and method perform real-time signal analysis of a specified EEG rhythm, extract frequency-domain phase information to estimate the next occurrence of a desired EEG rhythm phase, and trigger a brain stimulation pulse so as to align precisely with this predicted EEG phase.

### CROSS REFERENCE TO RELATED APPLICATIONS

This application claims the benefit of and priority to U.S. Provisional Patent Application Ser. No. 62/109,982, filed Jan. 30, 2015, which is incorporated herein by reference in its entirety.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### REFERENCE TO A SEQUENCE LISTING, A TABLE, OR A COMPUTER PROGRAM LISTING APPENDIX

Not Applicable.

### STATEMENT REGARDING PRIOR DISCLOSURES BY THE INVENTOR OR A JOINT INVENTOR

Not Applicable.

### BACKGROUND OF THE INVENTION

1. Field of the Invention

This invention relates generally to a device and method of phase-locking brain stimulation to electroencephalographic rhythms, and more particularly to a device and method that utilize real-time EEG analysis to measure a patient's brain state, and determine the most opportune time points at which to administer brain stimulation for maximum therapeutic benefit.

2. Description of the Related Art

Methods of non-invasive brain stimulation allow human function to be altered to treat neurological disorders without the need for costly and dangerous neurosurgeries and with greater specificity and precision than neuropharmacological interventions. Such methods of non-invasive human brain stimulation include transcranial magnetic stimulation (TMS), repetitive TMS (rTMS), transcranial direct current stimulation (tDCS) and transcranial electrical stimulation (TES). These methods of non-invasive brain stimulation use magnetic fields or small electrical currents to alter neuroplasticity of neural circuits within the human brain. Clinical neurology and psychiatric research have shown these methods to be safe and cost-effective therapeutic tools for a number of psychiatric disorders (e.g., depression, schizophrenia, and obsessive-compulsive disorder) as well as for rehabilitation of brain function following brain injury, aneurysm, stroke, or degenerative neurological disease (e.g., epilepsy, Parkinson's, Alzheimer's or Tourrette's).

TMS stimulates the human brain noninvasively using a magnetic stimulating coil to produce very strong but very brief magnetic fields that, when placed against the scalp, penetrate unimpeded and relatively painlessly through the tissues of the head into brain tissue of the patient to induce neural activity in brain areas directly beneath the stimulating coil. rTMS manipulates states of cortical neuroplasticity through repeated delivery of TMS pulses in a particular temporal pattern. The application of rTMS at high frequencies (≧5-Hz) induces a prolonged state of cortical excitability that endures well beyond the stimulation period, and conversely, rTMS at low frequencies is inhibitory, inducing reductions in cortical excitability. Effects of rTMS can persist for 30 to 40 minutes and occur as a result of NMDA-dependent mechanisms of LTP (excitatory rTMS) or LTD (inhibitory rTMS).

Though therapeutic uses of brain stimulation are expanding, improvements in the efficacy of these techniques have remained relatively stagnant over the past decade. One improvement to the effectiveness of brain stimulation has been the integration of neuroimaging measures, namely an electroencephalogram (EEG), to non-invasively monitor and record specific brain wave patterns in a patient from surface electrodes affixed to the scalp. EEG provides a time-resolved measure of population-level activity and forms a convenient method by which to quantify and identify transient states of brain activity that are ideal for administering brain stimulation pulses for maximum therapeutic benefit.

A major limitation of these current brain stimulation therapies, however, is that they are all administered without regard to the natural, continuous, and ongoing fluctuations in the activity of a patient's brain.

Therefore, it is desirable to improve the efficacy of non-invasive brain stimulation as a therapeutic tool by incorporating neuroimaging measures of brain activity in order to determine the most advantageous time points at which to administer brain stimulation form maximal therapeutic effect.

It is further desirable to provide a device and method of phase-locking brain stimulation to electroencephalographic rhythms.

It is yet further desirable to provide a device and method that utilize real-time EEG analysis to measure a patient's brain state, and determine the most opportune time points at which to administer brain stimulation for maximum therapeutic benefit.

It is still further desirable to provide a device and method that use measurements of sub-second fluctuations in brain activity (brain microstates) to estimate ideal time points at which to administer brain stimulation pulses for maximum therapeutic benefit to the patient.

It is still yet further desirable to provide a device and method of phase-locking brain stimulation to electroencephalographic rhythms that allows a patient's EEG to be monitored in order to determine the most advantageous times to deliver a TMS or TES pulse to the patient.

Other advantages and features of the invention will be apparent from the following description and from the claims.

### BRIEF SUMMARY OF THE INVENTION

In general, in a first aspect, the invention relates to a brain stimulation electroencephalogram phase-locking device having a brain stimulation electroencephalogram phase-locking unit in communication with an electroencephalogram input configured to be in communication with an electroencephalogram using an analog-to-digital conversion unit. A brain stimulation pulse trigger output is in communication with the phase-lock unit and configured to be in communication with a brain stimulation device. The phase-locking unit includes an EEG data preprocessing module, an EEG time-frequency analysis module and a predictive phase of EEG rhythm module. The brain stimulation device is configured to provide transcranial magnetic stimulation, repetitive transcranial magnetic stimulation, transcranial direct current stimulation or transcranial electrical stimulation.

In addition, the phase-locking device may include an electroencephalogram amplifier in communication with the electroencephalogram input and the electroencephalogram. The brain stimulation electroencephalogram phase-locking unit can include a recording unit in communication with the electroencephalogram input and an analysis unit in communication with the recording unit and the brain stimulation pulse trigger output. Further, the brain stimulation pulse trigger output can include a data acquisition output in communication with a microcontroller, and the microcontroller in communication with a transistor-transistor logic trigger of the brain stimulation device.

In general, in a second aspect, the invention relates to a system for phase-locking brain stimulation to electroencephalographic rhythms. The system includes an electroencephalogram-gated, transcranial magnetic stimulation device configured to provide a timed brain stimulation pulse to a transcranial magnetic stimulation coil generally in synchrony with a predetermined phase of an endogenous brain rhythm as measured by an electroencephalogram. The electroencephalogram-gated, transcranial magnetic stimulation device can include a recording unit in communication with the electroencephalogram for recording the endogenous brain rhythm and an analysis unit in communication with the recording unit and the transcranial magnetic stimulation coil. The analysis unit may have an electroencephalogram data preprocessing module, an electroencephalogram time-frequency analysis module and a predictive phase of electroencephalogram rhythm module. In addition, the analysis unit may be configured to perform real-time signal analysis of the endogenous brain rhythm, extract frequency-domain phase information to estimate a predicted electroencephalogram rhythm phase, and trigger the timed brain stimulation pulse in synchronicity with the predicted electroencephalogram rhythm phase.

In general, in a third aspect, the invention relates to a method for phase-locking brain stimulation to electroencephalographic rhythms. The method includes (1) acquiring an endogenous brain rhythm signal, such as an alpha brain rhythm signal, from a patient from an electroencephalogram; (2) performing real-time or near real-time signal analysis of the endogenous brain rhythm signal; (3) extracting frequency-domain phase information from the endogenous brain rhythm to estimate a predicted electroencephalogram rhythm phase; and then (4) triggering a brain stimulation pulse or pattern of pulses in alignment with the predicted electroencephalogram phase. The frequency-domain phase information may be extracted from the alpha brain rhythm to estimate an predicted alpha rhythm phase of the patient.

The method may also include aligning a repetitive transcranial magnetic stimulation pulse to particular phases of the predicted alpha rhythm phase of the patient, and triggering the repetitive transcranial magnetic stimulation pulse in alignment with the predicted alpha rhythm phase of the patient.

Moreover, the method may include (1) extracting an oscillatory phase of a predetermined frequency or frequency band from the endogenous brain rhythm to estimate a predicted predetermined rhythm phase of the patient; (2) aligning a repetitive transcranial magnetic stimulation pulse to the predetermined frequency or frequency band; and then (3) triggering the brain stimulation pulse further comprises the step of triggering the repetitive transcranial magnetic stimulation pulse in alignment with the predicted predetermined rhythm phase of the patient.

### BRIEF DESCRIPTION OF THE DRAWINGS

FIG. 1(A) illustrates an example of a device and method for phase-locking brain stimulation to electroencephalographic rhythms in accordance with an illustrative embodiment of the invention disclosed herein;

FIG. 1(B) graphically illustrates an example of aligning each of the brain stimulation pulses to a desired phase of an EEG rhythm so that the pulses interact with the ideal brain states to maximize the therapeutic benefits of the invention disclosed herein;

FIG. 1(C) illustrates exemplary hardware and software components of a device and method for phase-locking brain stimulation to electroencephalographic rhythms in accordance with an illustrative embodiment of the invention disclosed herein;

FIG. 2 illustrates an example of a device and method for alpha phase-locked rTMS, with the phase alignment of high-frequency (50 Hz) bursts of rTMS;

FIG. 3 illustrates an example of a real-time EEG analysis setup in accordance with an illustrative embodiment of the invention disclosed herein;

FIG. 4 graphically illustrates alpha-phase locking of 50 Hz rTMS bursts (Cz) in accordance with an illustrative embodiment of the invention disclosed herein;

FIG. 5 graphically illustrates potentiation of MEP modulation by alpha phase-locked rTMS in accordance with an illustrative embodiment of the invention disclosed herein;

FIG. 6 graphically illustrates potentiation of TEPs by alpha phase-locked rTMS (FC2) in accordance with an illustrative embodiment of the invention disclosed herein;

FIG. 7 illustrates MEPs and TEPs in accordance with an illustrative embodiment of the invention disclosed herein.

FIG. 8 illustrates an example of a session timeline in accordance with an illustrative embodiment of the invention disclosed herein.

### DETAILED DESCRIPTION OF THE INVENTION

The devices and methods discussed herein are merely illustrative of specific manners in which to make and use this invention and are not to be interpreted as limiting in scope.

While the invention has been described with a certain degree of particularity, it is to be noted that many modifications may be made in the details of the construction and the arrangement of the elements and components of the devices and/or in the sequences and steps of the methods without departing from the scope of this disclosure. It is understood that the devices and methods are not limited to the embodiments set forth herein for purposes of exemplification.

The invention is generally directed to a device and method for phase-locking brain stimulation to electroencephalographic rhythms, which improves the accuracy, specificity, and effectiveness of non-invasive brain stimulation devices by timing pulses of brain stimulation to occur in synchrony with naturally occurring brain rhythms measured at the scalp of a patient in order to treat an assortment of neurological and psychiatric conditions. The device and method provided herein improve non-invasive brain stimulation techniques by time-locking the onset of brain stimulation to the phase of naturally-occurring rhythmic oscillations of brain activity that can be recorded with electroencephalography (EEG). The device and method perform real-time signal analysis of a specified EEG rhythm, extract frequency-domain phase information to estimate the next occurrence of a desired EEG rhythm phase, and trigger a brain stimulation pulse so as to align precisely with this predicted EEG phase.

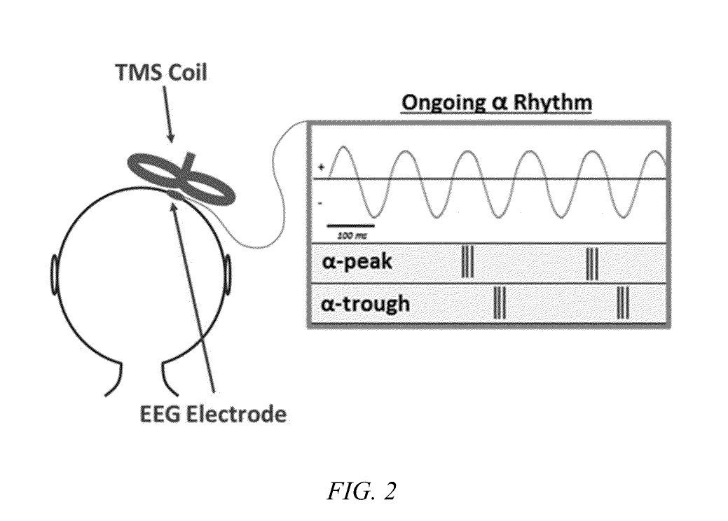

The device and method for phase-locking brain stimulation to electroencephalographic rhythms utilize an EEG gated method of brain stimulation based on measures of the phase of endogenous brain rhythms. More particularly, the device and method uses EEG-gated rTMS in order to trigger pulses of an rTMS protocol to align with particular phase of an endogenous brain rhythm, namely the alpha rhythm. (FIG. 2) Alpha band oscillations are ubiquitous across cortex and form an inhibitory rhythm that drives cortical tissue between synchronized cycles of inhibition, fluctuating between states of maximum and minimum inhibition. Brain rhythms are large-scale oscillations of neural activity in the human brain, measured from surface electrodes on the scalp using EEG at a particular frequency or within a particular frequency band; for example, the human alpha rhythm is an EEG oscillation within a 7.5 to 12.5 Hz band. Brain rhythms occur across numerous frequency bands and reflect oscillations between states of excitability, arousal, cognitive, perceptual, motor, or emotional in brain circuits, and the phase of brain rhythms is linked to a cycle of activity between the balance of excitatory and inhibitory brain activity.

The device and method of the present application align the onset of brain stimulation to a particular phase of a targeted brain rhythm in order to increase the therapeutic benefits of brain stimulation. For example, as discussed below, using the invention to align magnetic pulses from a TMS brain stimulator to particular phases of the human alpha rhythm resulted in a several-fold increase in a standard measure of TMS efficacy in the motor system.

The device and method include an automated EEG-brain stimulation interface that performs continuous real-time analysis of EEG phase to estimate future time points to trigger aligned in time to a specified phase. The device and method analyzes, the EEG data in real-time, extracts the oscillatory phase of a pre-specified frequency (or frequency band), estimates the next time of occurrence of a target EEG phase, and then outputs a signal to trigger a brain stimulation pulse (or pattern of pulses) at that predicted time.

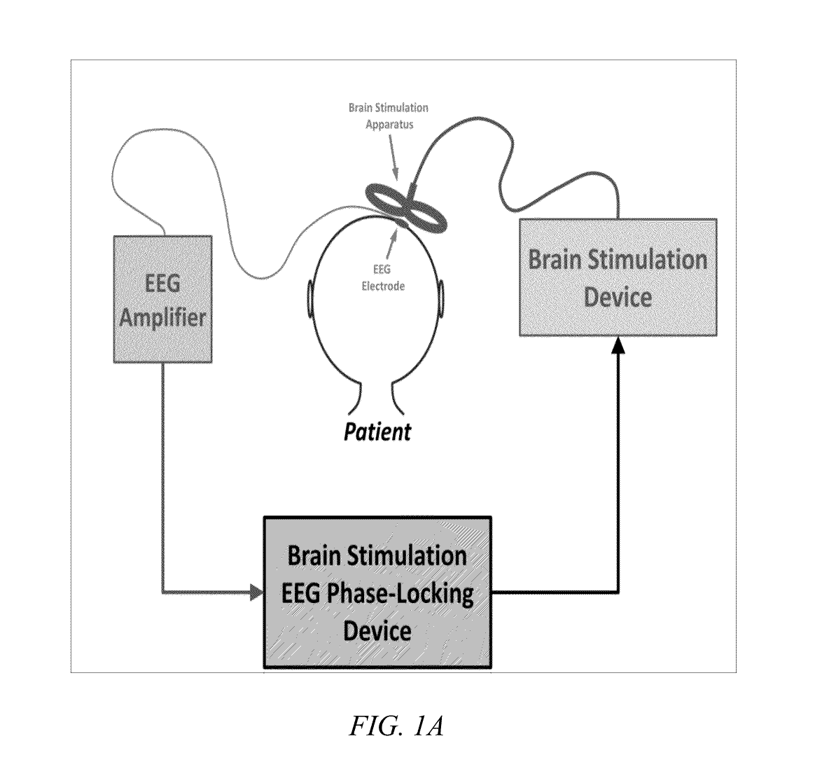

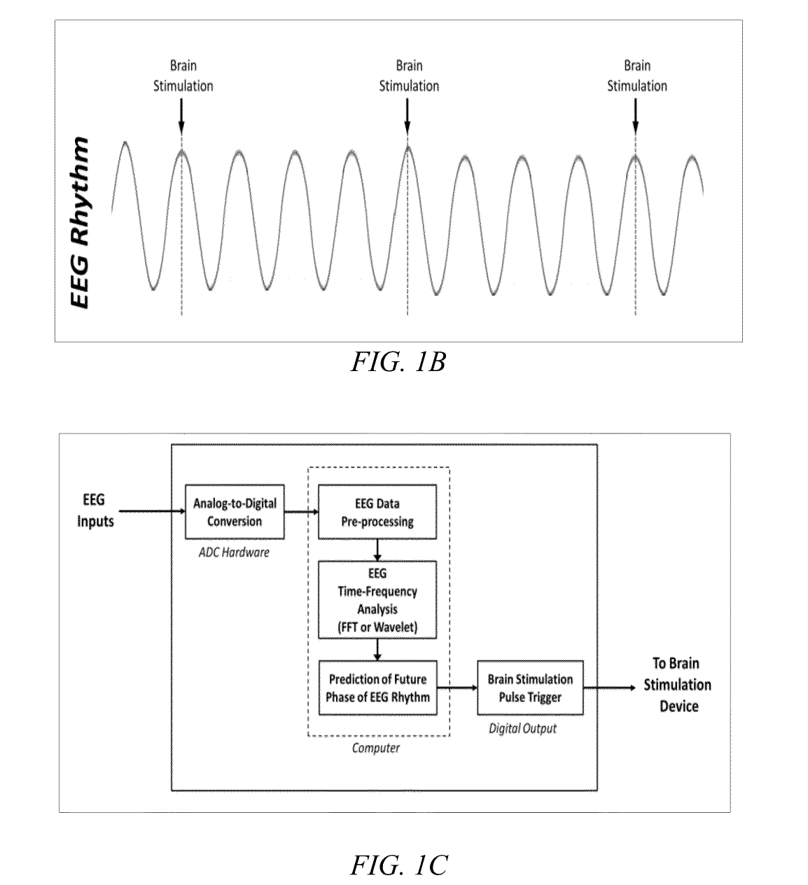

Referring now to FIG. 1(A), the device and method for phase-locking brain stimulation to electroencephalographic rhythms serves as an interface between EEG recordings and brain stimulation administration, acquiring ongoing EEG from an amplifier and analyzing this EEG data to estimate when brain stimulation should be administered so as to align to a specified phase of a specified brain rhythm. As illustrated in FIG. 1(B), the device and method perform continuous time-frequency analyses on segments of EEG data to estimate a future time point at which a triggered brain stimulation event should be triggered to align with a specified EEG brain rhythm phase. Moreover, the device and method for phase-locking brain stimulation to electroencephalographic rhythms continuously acquires data from an existing EEG amplifier, and as this data is acquired, its phase information is extracted from a pre-specified brain rhythm (i.e., a particular frequency or frequency band). The extracted phase information is then used to quantify the brain rhythm's phase over a short time interval then estimate the session of brain stimulation therapy administers hundreds of brain stimulation pulses. The device and method then align each of these pulses to a desired phase of an EEG rhythm (FIG. 1(B)) so that brain stimulation pulses interact with ideal brain states to maximize therapeutic benefit. The inventive device and method provide for flexible specification of brain rhythm frequency and phase in order to address various neurological and psychiatric disorders that require different brain rhythms and/or phases to be targeted.

As exemplified in FIG. 1(C), the device may receive inputs from the electrodes of an EEG amplifier and converts the analog signals to a digital representation using analog-to-digital conversion (ADC) hardware. The digitized EEG then goes through several optional steps of data pre-processing, specified by the user (e.g., re-referencing, filtering, Laplacian transform). Following the data preprocessing step, frequency-domain and phase-domain information of ongoing EEG is extracted by a moving fast-Fourier transform (FFT), and the continuously extracted frequency and phase information are used to estimate a future time-of-phase. The device and method trigger a brain stimulation event at the estimated time by outputting a digital signal in the form of a transistor-transistor logic (TTL) pulse to a brain stimulation device.

The inventive device allows a user to specify: (1) target electrode(s), specification of which EEG amplifier channel (or channels) to use for analysis; (2) target frequency range, the desired EEG brain rhythm (frequency) range within which to analyze and trigger brain stimulation events; (3) target phase, the desired EEG brain rhythm phase to align brain stimulation; (4) analysis window, the time interval over which to perform frequency and phase-domain analyses; (5) window shift, t time by which to shift each FFT; (6) EEG rhythm threshold, the minimum frequency-domain power allowed for phase aligned brain stimulation to be triggered; (7) cycles/time to wait, the number of cycles of EEG oscillation (or time) to wait before alignment of brain stimulation to EEG phase (for use in calculation of future time-of phase estimation); (8) peak or mean phase, specification to use the maximum or mean phase within the target frequency range; (9) pre-processing steps, the selection of preprocessing steps to perform on EEG data; and/or (10) output trigger, specification of the output signal to be sent to the brain stimulation device (number of pulses and duty cycle of these pulses).

In addition, the device and method for phase-locking brain stimulation to electroencephalographic rhythms may include a “simulation mode” in order to perform a simulated analysis and protocol (without triggering any actual brain stimulation) to estimate phase-locking accuracy in advance of brain stimulation administration.

The device and method for phase-locking brain stimulation to electroencephalographic rhythms leverages information about ongoing brain activity to improve the efficacy and specificity of brain stimulation therapies for neurology and psychiatry applications. The device and method for phase-locking brain stimulation to electroencephalographic rhythms improves treatment effects, durations, and outcomes, and also makes previously impractical brain stimulation therapies clinically viable as well as opening new avenues of brain stimulation treatments.

### EXAMPLES

The device and method for phase-locking brain stimulation to electroencephalographic rhythms disclosed herein is further illustrated by the following examples, which are provided for the purpose of demonstration rather than limitation. The following examples illustrated the improved efficacy of rTMS-induced neuroplasticity by gating TMS pulses according to continuous measurement of the instantaneous phase of the endogenous alpha rhythm using the inventive device and method provided herein.

The examples used the human motor system as a model to determine the feasibility of the device and method, and as such, the results of the examples described below can be directly translated for use in motor neurorehabilitation (i.e., stroke, Parkinson's, dystonia). The device and method for phase-locking brain stimulation to electroencephalographic rhythms disclosed herein can be applied to a wide range of dysfunctional networks of the central nervous system, and given the ubiquity of the alpha rhythm across the human cerebral cortex, the device and method may be used for stimulation of other brain networks in order to improve rTMS as a therapeutic tool for a variety of neurological conditions (e.g., epilepsy, neurogenic pain, tinnitus) and psychiatric disorders (e.g., depression and schizophrenia).

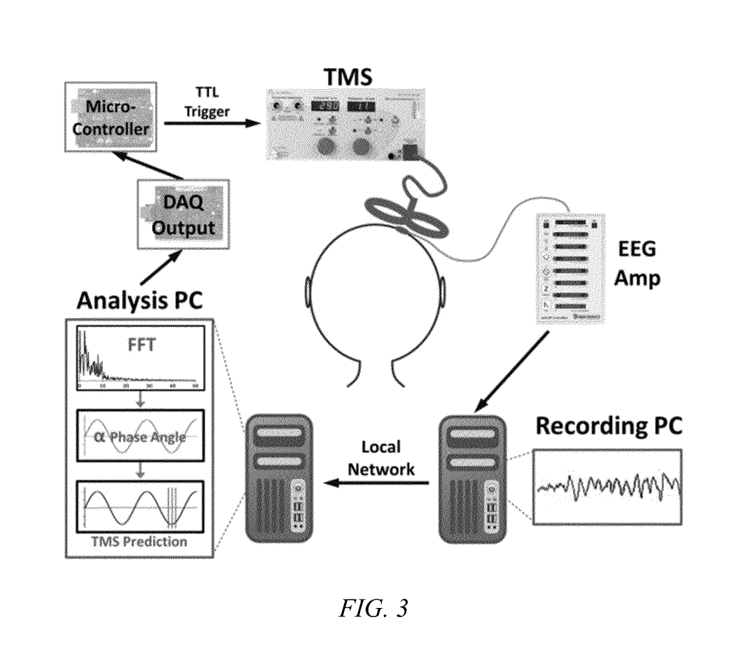

The examples illustrate the inventive alpha phase-locking to improve the efficacy of rTMS-induced neuroplasticity, using measures of cortical excitability from the motor system in healthy subjects as a model. The examples use real-time frequency-domain analyses on EEG to align pulses of an rTMS protocol to either the peak or trough phase of the alpha rhythm. Example 1 illustrates accuracy of experimental configuration on simulated signals and pilot subjects. Example 2 illustrates the interaction effects of alpha phase on excitatory rTMS by aligning high-frequency (50-Hz) bursts of rTMS to alpha phase (FIG. 3). Example 3 illustrates the effects of alpha phase on inhibitory rTMS by phase-locking low-frequency rTMS (˜1-Hz). Changes in cortical excitability are then assessed by pre- and post-rTMS measurement of motor-evoked potentials (MEPs), cortical TMS-evoked potentials (TEPs), response-locked event-related potentials (ERPs), and behavioral measurement of response times. Improvements in the efficacy of alpha phase-locked rTMS in Examples 2 and 3 are then determined by comparison to conventional intermittent theta-burst stimulation (iTBS) and 1-Hz rTMS, respectively.

### General Methodology of Examples:

Subjects:

Sixteen right-handed subjects were recruited for each experiment (32 total) from the University of Arkansas undergraduate and graduate population. Subjects participated in four experimental sessions conducted on separate days. Each session was three to four hours in duration. Subjects were compensated $15.00/hour for their participation. Prior to participation, subjects were screened extensively for safety with TMS.

EEG and EMG Recording:

EEG was recorded with a 64-channel BrainAmp DC (Brain Products, Munich, Germany). This amplifier is specially designed for use with TMS and is capable of recovering from magnetic artifact within 10-20 ms. EEG was recorded from low-profile electrode caps with notched Ag/AgCl ring electrodes placed according to the modified 10-20 system. Continuous EEG was recorded relative to the left mastoid. Bipolar electrode pairs above and below the right eye and on the outer canthus of each eye record the vertical and horizontal electro-oculogram (EOG), respectively. Electrode impedance was kept at or below 5 kΩ. Data was recorded at 5000 Hz and analog filtered at acquisition between DC and 1000 Hz (half-amplitude cutoffs). Offline, scalp-recorded EEG was arithmetically re-referenced to the average of left and right mastoids.

EMG was recorded simultaneously with EEG from the left and right hands using a 16-channel BrainAmp ExG bipolar amplifier (Brain Products, Munich, Germany). Bipolar Ag/AgCl electrodes was positioned in a belly-tendon arrangement on the first dorsal interosseous muscle of the left and right hand with ground electrode positioned on the left wrist. Electrode impedance was kept at or below 15 kΩ. Data was recorded at 5000 Hz (DC—1000 Hz). Offline, EMG data was digitally band-pass filtered from 10 to 500 Hz (24 dB/oct). MEPs were formed from EMG segments −20 to 60 ms. Segments were baseline corrected and rejected if they contained EMG exceeding an absolute voltage of 50 μV in the baseline or an MEP of less than 100 μV in magnitude.

For calculation of TMS-evoked potentials (TEPs), data points containing the TMS artifact were discarded and interpolated with a 4th-order B-spline to avoid digital filtering distortions. Continuous EEG data was then band-pass filtered between 1.0 and 50 Hz (24 dB/oct) and segmented into epochs of −100 to 300 ms relative to TMS pulse onset. Segments were baseline corrected according to the 100 ms pre-stimulus interval. Any segment with an absolute voltage exceeding 100 μV in any EEG or EOG channel was discarded to remove trials containing amplifier saturations, electrical, muscular, or ocular artifacts.

Analysis of event-related potentials in the motor task involved digital filtering of EEG (0.1-30 Hz; 24 dB/oct) and EMG (10-500 Hz; 24 dB/oct), and then forming time-locked segments relative either to EMG onset (simple response time task) from −400 to 200 ms or stimulus onset (go-no go task) from −100 to 600 ms. All segments were baseline corrected according to a −100 ms pre-stimulus interval. Segments containing voltage exceeding ±100 μV was rejected as artifacts. Average readiness potentials in each motor task were calculated separately for left and right hands.

Magnetic Stimulation:

TMS was administered using a PowerMag 100 stimulator (Brain Products, Munich, Germany) with a figure-of-eight coil in a posterior-to-anterior orientation. Representations of right M1 hand representations were mapped out according to a standard grid method. All TMS was administered to the hand representation of right M1. This site of stimulation was chosen to investigate effects of alpha-phase locking as it is not as sensitive as left M1 in right-handers and thus made the findings more generalizable to other regions of cortex. To account for subject variability in TMS sensitivity, stimulation intensity was set to each subject's active or resting motor threshold. Resting motor threshold (RMT) was determined as the stimulator intensity required to obtain a motor evoked potential on half of trials with the hand kept stationary. RMT was measured using a 1-up 1-down staircase (12 reversals). Active motor threshold (AMT) was measured in the same manner as RMT but with the hand contracted at 10% maximum grip force. A dynamometer was used to give continuous visual feedback of force output during AMT measurement. High-frequency (50 Hz) bursts of TMS was given at an intensity of 80% AMT whereas low-frequency rTMS (1 Hz) was given at 90% AMT. Single-pulse TMS (MEPs and TEPs) was given at 110% RMT. Single-pulse TMS was also administered to left and right M1 whereas rTMS was always administered to right M1.

Alpha Phase-Locked rTMS:

The device and method provided herein perform real-time EEG analysis and output TMS pulses at precise times to align with alpha phase (FIG. 2). The device and method utilize a flexible system of custom software and hardware to perform real-time phase-locked rTMS administration (FIG. 3). The system streams ongoing EEG from a data recording device over a local network connection to an analysis device for data processing. The analysis device may runs a set of custom Matlab® scripts which segments EEG, performs a FFT on segments, determines the dominant frequency within a frequency band (alpha), then extracts the phase angle of this frequency. This phase information is then used to compute the temporal delay until the next peak or trough. The analysis device incorporates this timing along with a measure of computational and data transmission delays to estimate the time at which a TMS pulse should be delivered to align with a peak or trough of the measured rhythm. The analysis device pauses for this delay then sends an output trigger via a data acquisition card to a microcontroller which in turn sends microsecond-accurate TTL pulses to the TMS device to trigger a pulse.

### Example 1

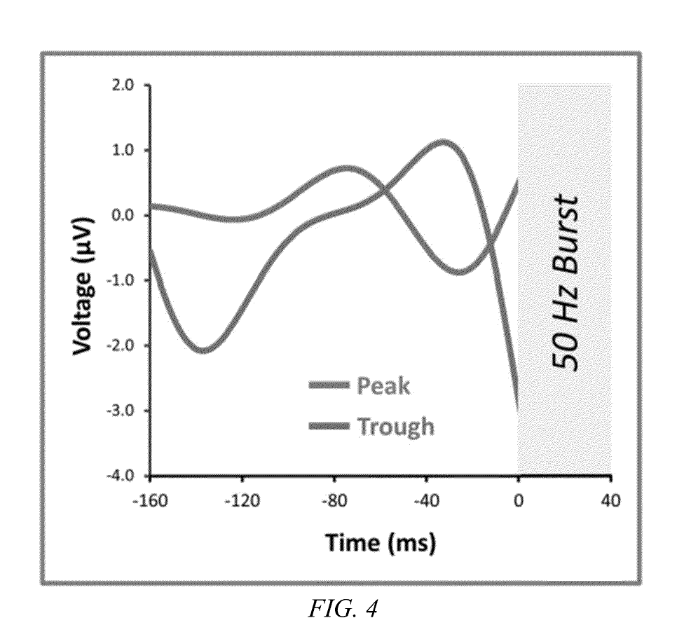

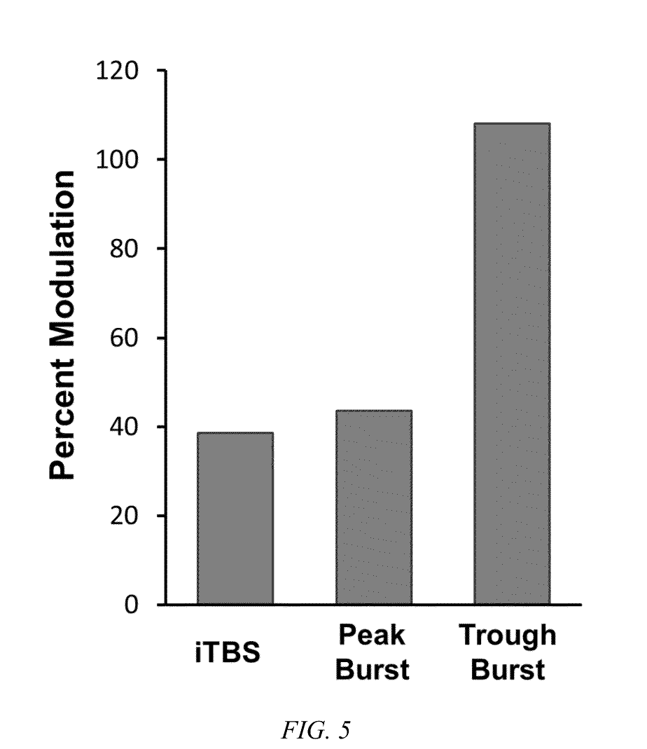

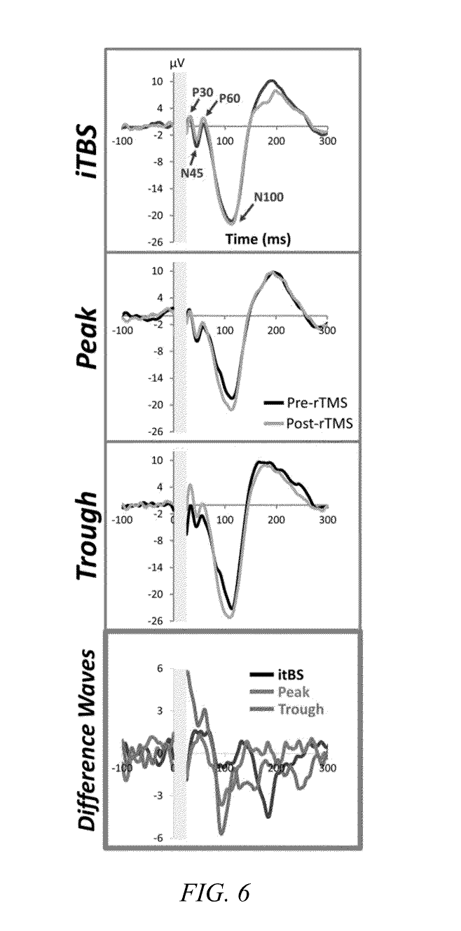

For Example 1, pilot data (N=4) was collected from peak burst, trough burst, and conventional iTBS sessions. MEP and TEP data for pre-rTMS and one post-rTMS time point directly following rTMS administration (equivalent to the pre-rTMS and 10-minute post-rTMS measures of MEPs and TEPs) was collected, and plots of time-domain averages of EEG activity within a short time window leading up to a 50 Hz burst of TMS pulses are illustrated in FIG. 4, plotted separately for peak burst and trough burst conditions. Example 1 shows the intended phase differences and temporal alignment for these two rTMS stimulation protocols. The 50-Hz burst of three pulses onset at an average phase of 16.8° for peak burst and 159.4° for trough burst. The data from Example 1 demonstrates the feasibility and effectiveness of the device and method disclosed herein for time-locking to a desired alpha phase. Percent modulation of MEPs for peak burst, trough burst, and conventional iTBS is presented in FIG. 5, and modulation of MEPs following conventional iTBS and peak burst rTMS are on the order of 30 to 40% whereas trough burst rTMS reveals a much larger modulation of 110%. A similar pattern can be seen across TEP components P30, N45, P60, and N100. Though each of these components is increased following active rTMS to right M1, these TEP components are much larger in amplitude following trough burst rTMS as compared to peak burst or conventional iTBS. These results of Example 1 demonstrate that phase alignment of high-frequency rTMS bursts to alpha trough per the invention of the present application significantly improves the efficacy of rTMS administration.

### Example 2

Example 2 addresses alpha phase and excitatory rTMS in order to determine the relationship between alpha phase and TMS-induced excitation in the human cortex. Alpha oscillations reflect cycles of inhibition in cortex, and as such, the instantaneous phase of alpha reflects the present state of excitability in cortex as it relates to the naturally occurring synchronous networks reflected in alpha. As a subpopulation of neurons within cortex is expected to be synchronously activated (or deactivated) according to alpha phase, inducing a state of neuroplasticity with TMS at a particular phase in order to provide a method of enhancing LTP within a target population of cells and/or modulating inter-regional communication. Alpha phase-related effects of TMS-induced excitability are examined by time-locking high-frequency bursts of TMS to peaks or troughs of alpha phase and comparing pre- and post-measures of cortical excitability from MEPs, TEPs, and ERPs. Example 2 compares the magnitude of phase-locked rTMS administration to a standard iTBS sequence and a sham condition to determine relative improvement in efficacy (or lack thereof) yielded by alpha phase-locking.

Procedure.

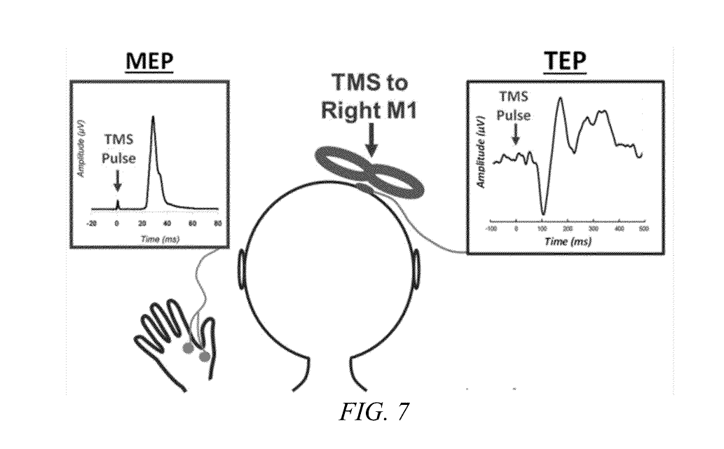

Subjects (N=16) participated in four sessions conducted on separate days. EEG and EMG were positioned, and the hand representations of left and right primary motor cortex was mapped and marked on the EEG cap. To minimize the introduction of noise and interference by the TMS coil into M1-proximal electrodes, a small thin foam patch was fastened to the electrode cap over these sites. RMT and AMT were then determined for left and right M1. Prior to rTMS protocol administration, behavioral and neurophysiological measures of baseline cortical excitability were taken. Subjects first performed a simple response time task. Subjects cued to respond with either the left or right hand (‘L’ or ‘R’), 2000 to 3000 ms later a black circle flashed briefly in the center of the screen (10% catch trials). Subjects responded as quickly as possible to the onset of this stimulus. Response times and readiness potentials obtained from EEG recordings were of interest in these measures. A second set of pre-rTMS measures was TMS-evoked responses: MEPs and TEPs (FIG. 7). Both of these measures were evoked by single pulses of TMS (110% RMT) to M1 at a random delay between 3000 and 5000 ms. Subjects were given four blocks of 40 trials, alternating between left and right M1 (order counterbalanced between subjects). MEPs were provided a standard and reliable measure of cortico-spinal excitability. TEP components provided a direct measure of cortical excitability and permitted a more detailed and time-resolved measurement of induced neuroplasticity.

Following pre-rTMS measures of cortical excitability, individual alpha frequency (IAF) was determined by measuring the dominant frequency in the alpha band (7.5-12.5 Hz) during a 2-minute fixation period. IAF was used as an anchor frequency to account for individual differences and determine the appropriate window of alpha band to use for triggering phase-locked TMS. This window was set as ±1.5 Hz around the measured IAF. For example, if IAF were 10.0 Hz, alpha phase-locking of TMS would be calculated according to the phase of the frequency of maximum power between 8.5 and 11.5 Hz.

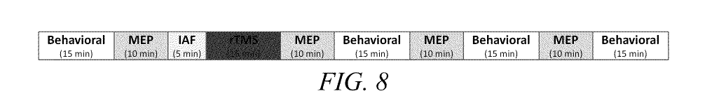

Once IAF had been determined, the session's rTMS protocol was administered: peak burst, trough burst, conventional iTBS, or sham. Peak burst rTMS consisted of bursts of three 50 Hz TMS pulses (80% AMT) centered on the positive peak of alpha phase. After each 50 Hz burst, a period of 300 ms was passed after which time the next 205 ms of EEG was segmented and used to estimate the onset of the next burst. A total of 200 such bursts were administered in trains of 10 bursts. In accord with current safety guidelines, a rest period of 8000 ms was given between each train. In the trough burst session, 50 Hz bursts were administered in the same manner but aligned to the estimated alpha trough rather than the peak. In the conventional iTBS session, rTMS was administered according to a standard iTBS protocol with three 50-Hz bursts (80% AMT) given every 200 ms in 20 trains of 10 bursts. The sham session also administered a standard iTBS protocol but with the TMS coil tilted at a 90° angle relative to the scalp, causing the magnetic field of the TMS pulse to be emitted into the open air rather than stimulating motor cortex. Each subject received each of these four protocols across four separate sessions. The order of rTMS protocols was counterbalanced between subjects according to a Latin square. Following administration of the session's rTMS protocol, measures of cortical excitability was repeated three times (FIG. 8).

Analyses.

MEPs were analyzed by first converting post-rTMS MEP amplitudes (peak-to-peak) into a measure of percent modulation relative to pre-rTMS amplitude. Percent modulation data was submitted to a 4×3 repeated-measure ANOVA with factors of session (peak, trough, iTBS, or sham) and time point (10, 30, or 50 minutes post-rTMS). Degrees of freedom were Huyn-Feldt corrected for violations of sphericity. Peak burst, trough burst, and iTBS all exhibited enhanced MEP amplitudes but trough burst rTMS showed significantly larger enhancements that persist for longer durations.

Analysis of TEPs focused on four components: P30, N45, P60, and N100. Each of these components can be reliably evoked in motor TEPs and have been associated with modulations in cortical excitability. Examining these components individually yielded valuable information as to the changes in cortical neurophysiology associated with phase-locked high-frequency bursts of TMS. Separate 4×4 repeated measures ANOVAs was run with factors of session (peak, trough, iTBS, or sham) and time point (pre-rTMS; 10, 30, or 50 minutes post-rTMS). Each TEP component exhibited enhanced post-rTMS amplitude (as compared to sham) but trough burst rTMS was largest and persist over longer periods of time.

Response-locked readiness potential ERPs was analyzed by first forming lateralized potentials for each response hand then submitting average voltage in a 100 ms time window (−100 to 0 ms) to a 2×4×4 repeated measures ANOVA with factors of hand (left or right), session (peak, trough, iTBS, or sham), and time point (pre-rTMS; 10, 30, or 50 minutes post-rTMS). Response times were also analyzed in this way, and larger readiness potentials and faster response times for left-hand responses post-rTMS were observed, with significantly bigger effects with trough burst rTMS, and significantly smaller effects in peak burst rTMS.

### Example 3

Example 3 addressed alpha phase and inhibitory rTMS (1 Hz) and determined the relationship between alpha phase and TMS-induced inhibition in the human cortex. Inhibitory effects on cortical excitability induced in the human brain by 1-Hz rTMS result from NMDA-dependent mechanisms of LTD. Like LTP, the induction of LTD is also activity dependent, requiring suprathreshold levels of low-frequency stimulation. Thus, the inhibitory effects of 1-Hz rTMS would also be expected to interact with the level of synchronized cortical excitability, just as high-frequency would. Example 3 examined this possibility by administering a 1-Hz inhibitory rTMS protocol with TMS pulses time-locked to specific phases of the alpha rhythm (peak or trough). As in Example 2, Example 3 compares the efficacy of alpha phase-locked 1-Hz rTMS to a standard 1-Hz rTMS protocol and a sham stimulation session.

Procedure.

The procedure of Example 3 was identical to that of Example 2 above 1 with the exception of the rTMS protocol administered. The four sessions of rTMS protocols in Example 3 was peak 1-Hz, trough 1-Hz, conventional 1-Hz, or sham. Peak 1-Hz consisted of a single TMS burst (90% AMT) centered on the positive peak of alpha phase. After each pulse, a period of 750 ms passed after which time the next 205 ms of EEG was used to estimate the onset of the next pulse. A total of 900 such peak-aligned pulses were given to right M1 over a 15-minute period. In the trough 1-Hz session, single pulses of TMS were given in the same manner but aligned to the alpha trough. The conventional 1-Hz session administered 900 pulses of 1-Hz rTMS (90% AMT) preset in timing and without regard to ongoing EEG. The sham session gave the same conventional 1-Hz rTMS protocol but with the coil tilted at an angle of 90° relative to the scalp.

Analyses.

MEPs, TEPs, readiness potentials, and response times was analyzed identical to the approach described in Example 2. Observed effects of Example 3 are also identical to Example 2 but in the opposite direction. All active forms of 1-Hz stimulation reduced MEP amplitudes, TEP components, readiness potentials and slow response times; however, these inhibitory effects were greatest and persist for a longer period following trough 1-Hz rTMS.

Whereas, the devices and methods have been described in relation to the drawings and claims, it should be understood that other and further modifications, apart from those shown or suggested herein, may be made within the scope of the invention.

What is claimed is:

1. A brain stimulation electroencephalogram phase-locking device, comprising:
an electroencephalogram input configured to be in communication with an electroencephalogram;
an analog-to-digital conversion unit in communication with said electroencephalogram input;
a brain stimulation electroencephalogram phase-locking unit in communication with said analog-to-digital conversion unit; said phase-locking unit comprising an EEG data preprocessing module, an EEG time-frequency analysis module and a predictive phase of EEG rhythm module; and
a brain stimulation pulse trigger output in communication with said phase-lock unit and configured to be in communication with a brain stimulation device.

2. The phase-locking device of claim 1 further comprising an electroencephalogram amplifier in communication with said electroencephalogram input and said electroencephalogram.

3. The phase-locking device of claim 1 wherein said brain stimulation device comprises a brain stimulation device configured to provide transcranial magnetic stimulation, repetitive transcranial magnetic stimulation, transcranial direct current stimulation or transcranial electrical stimulation.

4. The phase-locking device of claim 1 wherein said brain stimulation electroencephalogram phase-locking unit further comprises a recording unit in communication with said electroencephalogram input and an analysis unit in communication with said recording unit and said brain stimulation pulse trigger output.

5. The phase-locking device of claim 1 wherein said brain stimulation pulse trigger output further comprises a data acquisition output in communication with a microcontroller, and said microcontroller in communication with a transistor-transistor logic trigger of said brain stimulation device.

6. A system for phase-locking brain stimulation to electroencephalographic rhythms, said system comprising:
an electroencephalogram-gated, transcranial magnetic stimulation device configured to provide a timed brain stimulation pulse to a transcranial magnetic stimulation coil generally in synchrony with a predetermined phase of an endogenous brain rhythm as measured by an electroencephalogram.

7. The system of claim 6 wherein said electroencephalogram-gated, transcranial magnetic stimulation device comprises:
a recording unit in communication with said electroencephalogram for recording said endogenous brain rhythm;
an analysis unit in communication with said recording unit and said transcranial magnetic stimulation coil.

8. The system of claim 7 wherein said analysis unit comprises an electroencephalogram data preprocessing module, an electroencephalogram time-frequency analysis module and a predictive phase of electroencephalogram rhythm module.

9. The system of claim 8 wherein said analysis unit is configured to perform real-time signal analysis of said endogenous brain rhythm, extract frequency-domain phase information to estimate a predicted electroencephalogram rhythm phase, and trigger said timed brain stimulation pulse in synchronicity with said predicted electroencephalogram rhythm phase.

10. A method for phase-locking brain stimulation to electroencephalographic rhythms, said method comprising the steps of:
acquiring an endogenous brain rhythm signal from a patient from an electroencephalogram;
performing real-time or near real-time signal analysis of said endogenous brain rhythm signal;
extracting frequency-domain phase information from said endogenous brain rhythm to estimate a predicted electroencephalogram rhythm phase; and
triggering a brain stimulation pulse in alignment with said predicted electroencephalogram phase.

11. The method of claim 10 wherein said step of acquiring said endogenous brain rhythm signal further comprises the step of acquiring a continuous alpha brain rhythm signal from said patient from said electroencephalogram.

12. The method of claim 11 wherein said step of extracting said frequency-domain phase information further comprises the step of extracting frequency-domain phase information from said alpha brain rhythm to estimate an predicted alpha rhythm phase of said patient.

13. The method of claim 12 further comprising the step of aligning a repetitive transcranial magnetic stimulation pulse to particular phases of said predicted alpha rhythm phase of said patient.

14. The method of claim 13 wherein said step of triggering said brain stimulation pulse further comprises the step of triggering said repetitive transcranial magnetic stimulation pulse in alignment with said predicted alpha rhythm phase of said patient.

15. The method of claim 14 wherein said repetitive transcranial magnetic stimulation pulse is a pattern of repetitive transcranial magnetic stimulation pulses.

16. The method of claim 10 wherein said step of extracting said frequency-domain phase information further comprises the step extracting an oscillatory phase of a predetermined frequency or frequency band from said endogenous brain rhythm to estimate a predicted predetermined rhythm phase of said patient.

17. The method of claim 16 further comprising the step of aligning a repetitive transcranial magnetic stimulation pulse to said predetermined frequency or frequency band.

18. The method of claim 17 wherein said step of triggering said brain stimulation pulse further comprises the step of triggering said repetitive transcranial magnetic stimulation pulse in alignment with said predicted predetermined rhythm phase of said patient.

19. The method of claim 18 wherein said repetitive transcranial magnetic stimulation pulse is a pattern of repetitive transcranial magnetic stimulation pulses.
